(19) United States
(12) Patent Application Publication
(10) Pub. No.: US 2004/0030239 A1
(43) Pub. Date: Feb. 12, 2004

(54) Method for MR/NMR imaging

(75) Inventors: Peter C.M. Van Zijl, Ellicott City, MD (US); Nicholas Goffeney, Baltimore, MD (US); Jeff H. Duyn, Kensington, MD (US); Jeff W.M. Bulte, Clarksville, MD (US)

Correspondence Address: EDWARDS & ANGELL. LLP, P.O. Box 9169, Boston, MA, 02209, US

(73) Assignee: The Johns Hopkins University School of Medicine

(21) Appl. No.: 10/319,375

(22) Filed: Dec. 13, 2002

Related U.S. Application Data
(60) Provisional application No. 60/339,668, filed on Dec. 13, 2001.

(51) Int. Cl. A61B 5/05

(52) U.S. Cl. 600/420

(57) ABSTRACT

The present invention features an MRI/NMR methodology or process for detecting exogenous amide protons in a region of interest of a body or sample via the water signal. Such methods and processes can be used for any of a number of purposes including determining and assessing the delivery and/or content of a molecular or cellular target(s), such as ligands, oglionucleotides, and RNA/DNA (including plasmids) tagged or labeled by an exogenous contrast agent sourcing such amide protons; detecting and assessing pH effects, more particularly the pH of the liquid pool (e.g., blood); and as a mechanism for MR/NMR signal enhancement (e.g., providing another mechanism for developing contrast between tissues, etc. of the region of interest.

This application claims the benefit of U.S. Provisional Application Serial No. 60/339,668 filed Dec. 13, 2001, the teachings of which are incorporated herein by reference.

### STATEMENT REGARDING FEDERALLY-SPONSORED RESEARCH

The present invention was supported by grants from the National Institute of Health (NCRR), grant number 5RO1 -RR11115 and the National Institute of Health (NINDS), grant number 5RO1-NS31490. The U.S. Government may have certain rights to the present invention.

### FIELD OF INVENTION

The present invention generally relates to apparatus and methods for magnetic resonance (MR) imaging (MRI), also known as nuclear magnetic resonance (NMR) imaging (NMRI). More particularly the present invention relates to methods for magnetic resonance imaging and spectroscopy relating to exchange of magnetization/saturation between protons and more specifically methods for detecting, assessing and imaging pH effects as well as methods for detecting, assessing and imaging delivery of a gene, cell, antibody or other molecular or cellular body to a specified organ or tissue in connection with a therapy or treatment therefore.

### BACKGROUND OF THE INVENTION

Atherosclerotic cardiovascular disease remains the leading cause of mortality in the United States (see, e.g., American Heart Association, 1999 Heart And Stroke Statistical Update, Dallas, Tex., American Heart Association). Gene therapy is a rapidly expanding field with great potential for the treatment of atherosclerotic cardiovascular diseases. Several genes, such as vascular endothelial growth factor (VEGF), have been shown to be useful for preventing acute thrombosis, blocking post-angioplasty restenosis, and stimulating growth of new blood vessels (angiogenesis) (Nabel, 1995, Circulation 91: 541-548; Isner, 1999, Hosp. Pract. 34: 69-74). However, precise monitoring of gene delivery into and expression from target atherosclerotic plaques is a challenging task. It should be recognized, however, that gene therapy also is considered in connection with treatment of a wide range of disorders and diseases such as for example, cancer and auto immune diseases and the like.

Recent in vitro studies have shown that gene expression in cell culture can be detected with imaging techniques, such as nuclear imaging (Tjuvajev, et al., 1995, Cancer Res. 55: 6126-61329; Yu, et al., 2000, Nature Medicine 6: 933-937), optical imaging (Contag, et al., 1998, Nat. Med. 4; 245-247; Yang, et al., 2001, Radiology 219(1): 171-5) and magnetic resonance (MR) imaging (Johnason, et al., 1993, Magn. Reson. Q. 9: 1-30: 13 14; Weissleder, et al., 2000, Nature Medicine 6: 351-354). This is important, for example, for detecting cancer, following the trajectory of drug delivery, insertion of genes functional gene expression, following stem cells in situ, etc.

Generally, delivery of nucleic acids in vivo has relied on forming complexes (e.g., via chemical bonds) between a contrast agent and a nucleic acid molecule (see, e.g., U.S. Pat. No. 6,232,295 B1; U.S. Pat. No. 6,284,220 B1) for purposes of providing a mechanism that facilitates or allows the imaging of the gene expression. For positron emission tomography (PET) and related technologies, radioactively labeled receptor ligands and cellular uptake comprises the contrast agent that provides the mechanism for tagging or labeling. As to magnetic resonance imaging, the contrast agents used have nuclear or relaxation properties for imaging that are different from the corresponding properties of the cells/tissue being imaged. Examples of MRI contrast agents include an imageable nucleus (such as ¹⁹F), radionuclides, diamagnetic, paramagnetic, ferromagnetic, superparamagnetic substances, iron-based contrast agents (e.g., iron-based agents include iron oxides, ferric iron, ferric ammonium citrate and the like), gadilinium-based contrast agents (e.g., gadolinium based contrast agents include diethylenetriaminepentaacetic (gadolinium-DTPA)), and manganese paramagnetic substances. Typical commercial MRI contrast agents include Omniscan, Magnevist (Nycomed Salutar, Inc.), and ProHance. Because such MRI contrast agents generally involve accumulation of metals within the body, particularly if the metal is released (i.e., no-longer bound) such accumulation of metals within the body increases the potential risk of toxicity.

Magnetic resonance imaging (MRI) is a technique that is capable of providing three-dimensional imaging of an object. A conventional MRI system typically includes a main or primary magnet that provides the main static magnetic field Bₒ, magnetic field gradient coils and radio frequency (RF) coils, which are used for spatial encoding, exciting and detecting the nuclei for imaging. Typically, the main magnet is designed to provide a homogeneous magnetic field in an internal region within the main magnet, for example, in the air space of a large central bore of a solenoid or in the air gap between the magnetic pole plates of a C-type magnet. The patient or object to be imaged is positioned in the homogeneous field region located in such air space. The gradient field and the RF coils are typically located external to the patient or object to be imaged and inside the geometry of the main or primary magnet(s) surrounding the air space. There is shown in U.S. Pat. Nos. 4,689,563; 4,968,937 and 5,990,681, the teachings of which are incorporated herein by reference, some exemplary MRI systems.

In MRI, high-resolution information is obtained on liquids such as intracellular or extra-cellular fluid, tumors such as benign or malignant tumors, inflammatory tissues such as muscles and the like through the medium of a nuclear magnetic resonance (NMR) signal of a nuclear magnetic resonance substance such a proton, fluorine, magnesium, phosphorous, sodium, calcium or the like found in the area (e.g., organ, muscle, etc.) of interest. In addition to being a non-invasive technique, the MRI images contain chemical information in addition to the morphological information, which can provide physiological information.

Most clinical uses of MRI of biological tissue, however, employ the water content and water relaxation properties to image anatomy and function with micro-liter resolution. The relaxation properties of water (¹H nuclei) are the basis for most of the contrast obtained by NMR imaging techniques. Conventional ¹H NMR images of biological tissues usually reflect a combination of spin-lattice (T1) and spin-spin (T2) water ¹H relaxation. The variations in water ¹H relaxation rate generate image contrast between different tissue and pathologies depending upon how the NMR image is collected.

With MRI based on ¹H water relaxation properties, the system typically detects signals from mobile protons (¹H) that have sufficiently long T2 relaxation times so that spatial encoding gradients can be played out between excitation and acquisition before the signal has completely decayed. The T2-values of less mobile protons associated with immobile macromolecules and membranes in biological tissues are too short (e.g., less than 1 ms) to be detected directly in the MRI process.

As has become known to those skilled in the art, however, coupling between the immobile, solid-like macromolecular protons and the mobile or “liquid” protons of water allows the spin state of the macromolecular protons to influence the spin state of the liquid protons through exchange processes. As is known in the art, it is possible to saturate the spins of the immobile, solid-like macromolecular protons (“immobile macromolecular spins”) preferentially using an off-resonance radio frequency (RF) pulse. The immobile macromolecular spins have a much broader absorption lineshape than the spins of the liquid protons (“liquid spins”), making them as much as 10⁶ times more sensitive to an appropriately placed off-resonance RF irradiation, as illustrated in FIG. 1. This saturation of the immobile, solid-like macromolecular spins can be transferred to the liquid spins, depending upon the rate of exchange between the two spin populations, and hence is detectable with MRI. This process also is typically referred to as magnetization transfer (MT) process. See also Magnetization Transfer in MRI: A Review; R. M. Henkelman, G. J. Stanisz and S. J. Graham; NMR Biomed 14, 57-64 (2001), the teachings of which are incorporated herein by reference in its entirety and U.S. Pat. No. 5,050,609, the teachings of which also are incorporated herein by reference in its entirety.

Magnetization transfer is more than just a probe into the proton spin interactions within tissues as it also provides a mechanism that can be used to provide additional advantageous contrast in MR images. One application for use of the magnetization technique is in magnetic resonance angiography (MRA). In MRA specific imaging sequences are used to suppress the signal from static tissues while enhancing signal from blood by means of inflow or phase effects. The signal contrast between the blood and other tissue can always be enhanced by using MT (which need not affect blood) to further suppress the background tissue signal. Better contrast between blood and tissue leads to better angiograms.

MRI of acute stroke is becoming an increasingly important procedure for rapid assessment of treatment options. Despite many available MRI modalities, it is presently difficult to assess the viability of the ischemic penumbra (i.e., a zone of reduced flow around the ischemic core). Also, impaired oxygen metabolism and concomitant pH changes are crucial in the progress of the ischemic cascade, however, pH effects cannot be ascertained using the water signal.

As is known to those skilled in the art, phosphorous (³¹P) magnetic resonance spectroscopy (MRS) can be used to assess pH, however, this particular technique has low spatial resolution (e.g., 20-30 ml) in part because the strength of the NMR signal from phosphorous is significantly less than that for the water signal. Phosphorous MRS, however, is not available on standard clinical equipment, which as noted above, is limited predominantly to those that use the water proton (¹H) signals. Also, given the time constraints usually involved with making timely diagnoses for purposes of treatment, such as for when dealing with acute stroke victims, it is not a practical option or practice to re-configure clinical equipment configured to use the water signal so it can perform phosphorous MRS to assess pH. Thus, detection of pH and assessment of pH effects cannot be practically performed in connection with the NMR imaging process.

In sum, it has become possible to use the water (¹H) signal in MRI for non-invasive assessment of functional and physiological parameters as well as for providing a mechanism for contrasting tissues being imaged. It has not been possible, however, to use this water signal for purposes of imaging pH effects.

There is found in, van Zijl et al., Magn. Reson. Med. 40:36-42 (1998), the use of NMR spectroscopy to measure pH of molecules such as glucose in vivo or ex vivo. The spectroscopic technique, however, is not used for MRI image acquisition nor are the compounds studied suitable for use in the visualization techniques of the present invention.

Balaban and co-workers have investigated exchange-based saturation-transfer effects and, by studying the reduction of the amplitude of the water signal, have been able to indirectly detect 5-100 mM concentrations of small molecules. However, such detection sensitivities are still several orders of magnitude below those achievable with contrast agents such as super-paramagnetic tags and laser-polarized noble gases. The noble gas contrast agents have shown the largest sensitivity enhancements ever reported for NMR, e.g., up to about 5 orders of magnitude increase in sensitivity for the signal of interest.

In general, Balaban reports small molecule (non-polymeric agents), and a certain dextran-type material, which is an oxygen-based polymer, not a nitrogen-based polymer. Balaban and coworkers have disclosed a metabolite detection technique for small molecule metabolites such as ammonia (Wolff and Balaban J. Mag. Res. 86:164-169 (1990)) including systems having water-macromolecule exchange (Guivel-Scharen et al., J. Magn. Reson. 133:36-45 (1998). The metabolite detection techniques measure the change in amplitude of the water proton signal as a function of metabolite concentration. Also, the molecules recited by Balaban can not be used to selectively bind to plasmids, DNA, oligonucleotides or recepetor ligands and further may not remain in the cell for a sufficiently long time for detection.

Balaban and coworkers have described another technique for chemical-exchange-dependent saturation transfer using a metal-free MRI contrast agent, but the contrast agents described in connection with this technique do not selectively bind cellular components such as DNA and receptor ligands and are of the type that frequently will diffuse from the target tissue or cell prior to detection. See Ward et al., J. Magn. Reson. 143:79-87 (2000) and the description of a patent application on file (http://wwwlssti.org/Digest/Tables/042800t.htm).

Efforts have been undertaken to develop exogenous contrast agents for pH detection via the water resonance. These techniques attempt to indirectly detect exchangeable protons through the water resonance in solution using such contrast agents. Discussions of such techniques are found in NMR Imaging of Labile Proton Exchange, S. Wolff and R. Balaban, JMR 86, p. 164 (1990); Detection of Proton Chemical Exchange Between Metabolites and Water in Biological Tissues, V. Guivel-Scharen, T. Sinnwell, S. D. Wolff and R. S. Balaban, J. Magn Reson 133, 36 (1998); A New Class of Contrast Agents for MRI Based Proton Chemical Exchange Dependent Saturation Transfer (CEST), K. M. Ward, A. H. Aletras and R. S. Balaban, J Magn Reson 143, 79 (2000); and K. M. Ward and R. S. Balaban, Determination of pH Using Water Protons and Chemical Exchange Dependent Saturation Transfer (CEST), Magn Reson Med 44(5): 799 (2000). The described exogenous contrast agents, however, are not suitable to selectively bind to plasmids, DNA, oligonucleotides or recpetor ligands.

It thus would be desirable to provide MRI methods embodying the use of non-metallic contrast agents to track and monitor the delivery and/or uptake of a molecular or cellular target(s), including but not limited to genes, gene expressions, stem cells and antibodies, using the water signal. In addition, it would be desirable to monitor pH, to detect pH, and to assess associated effects using the water signal and such non-metallic exogenous contrast agents. It would be particularly desirable to provide magnetic resonance imaging methods that would produce pH sensitive MRI contrast by exploiting for example the magnetization exchange between water protons and the amide protons of the exogenous contrast agents of the present invention. Further, it would be desirable to use such methods for monitoring, detecting and assessing pH in connection with treatment of brain related disorders and diseases, cardiac disorders and diseases, and cancer and to use such methods for monitoring, detecting and assessing pH in vivo and pathologically for any of a number of diseases or disorders of a human body, including but not limited to cancers, ischemia, Alzheimers and Parkinsons.

### SUMMARY OF THE INVENTION

The present invention features an MRI/NMR methodology or process for detecting exogenous amide protons in a region of interest of a body or sample via the water signal. Such methods and processes can be used for any of a number of purposes including determining and assessing the delivery and/or content of a molecular or cellular target(s), such as ligands, oglionucleotides, and RNA/DNA (including plasmids) tagged or labeled by an exogenous contrast agent sourcing such amide protons; detecting and assessing pH effects, more particularly the pH of the liquid pool (e.g., blood); and as a mechanism for MR/NMR signal enhancement (e.g., providing another mechanism for developing contrast between tissues, etc. of the region of interest. According to various aspects of the present invention, also featured are methods whereby assessment of the delivery or the efficacy of delivery, pH effects or the signal enhancement can be used in connection with diagnosis and treatment of any of a number of diseases or disorders of the body, including but not limited to, brain related disorders and diseases, cardiac diseases and disorders, cancer, ischemia, Alziheimers, Parkinsons, and auto-immune diseases.

According to one aspect of the present invention, there is featured a method for determining an effect of amide proton content and properties of an exogenous contrast agent on a water signal as measured by one of MRI or NMR spectroscopy or spectroscopic imaging. The exogenous contrast agent is configured and arranged so as to provide a pool of amide protons that is in exchange with another pool of protons. Such a method includes irradiating the pool of exogenous amide protons that is in exchange with said another pool of protons to label the amide protons of said pool of amide protons and measuring the effect on the protons the amide protons are in exchange with. The method further includes determining an amide proton transfer effect corresponding to the transfer of saturation between said pool of amide protons and said another pool of protons, and determining one of amide proton content, pH or pH effects from the determined amide proton transfer effect. In particular embodiments, the exogenous contrast agent comprises one of one of a cationic polymer, a polymide (e.g., dendrimers, poly-lysines and polyglutamate), polyimino, poly-amino, or polyimine compounds.

In further particular embodiments, the contrast agent comprises a polymer having a plurality of functional groups capable of exchanging at least one amide proton with water and the plurality of functional groups have a resonance frequency different from the resonance frequency of water and which can be saturated by proton exchange between the functional group and water. In other embodiments, the functional group has one of a pKₐ in the range of between about 3 and about 5, a pKₐ in the range of between about 3.5 and about 4.5 or a pKₐ of about 4. Also, the functional group is selected from primary amides, primary amines, secondary amines, imines, imides, mono functional ureas, 1,3-difunctional ureas and combinations thereof. In yet further embodiments, there is one of at least one exchangeable protons per monomer repeat unit of the cationic polymer, at least two exchangeable protons per monomer repeat unit of the cationic polymer, at least two (2) exchangeable protons per kDalton in the cationic polymer, at least four (4) exchangeable protons per kDalton in the cationic polymer or at least ten (10) exchangeable protons per kDalton in the cationic polymer.

The step of irradiating further includes irradiating the exogenous amide protons at a resonance in a proton spectrum of the amide protons, more particularly, irradiating the amide protons with electromagnetic radiation at about a 8.3 ppm resonance in a proton spectrum of the amide protons, more specifically irradiating the amide protons with electromagnetic radiation around a 8.3 ppm resonance in a proton spectrum of the amide protons. This also includes a range of about ±3-4 ppm surrounding the main amide resonance, where other amide resonances of mobile spectral components may resonate.

In further embodiments, such a method further includes establishing a relationship between proton transfer ratio and/or intensity of amide protons and said one of amide proton content, tissue pH or pH effects; more particularly establishing an empirical relationship between the proton transfer ratio of amide protons and said one of amide proton content, tissue pH or pH effects.

In an exemplary embodiment, said establishing an empirical relationship includes establishing an empirical relationship between the proton transfer ratio and/or intensity of amide protons and pH including: irradiating a first pool including amide protons of the contrast agent, that is in exchange with a second pool of protons, with sufficient electromagnetic radiation to label the amide protons of said first pool, determining a given amide proton transfer ratio corresponding to the transfer of saturation between said first pool of amide protons and said second pool of protons and performing a phosphorus spectroscopy to determine a pH value corresponding to the determined amide proton transfer rate. Said irradiating, determining and performing is repeated so as to generate a plurality of pH values corresponding to respective determined amide proton transfer ratios. Whereby the empirical relationship is created using the generated plurality of pH values corresponding to respective determined amide proton transfer ratios.

According to another aspect of the present invention, there is featured a method for magnetic resonance imaging comprising the steps of locating a contrast agent within a region of interest for a body or sample, the contrast agent being characterized as being a source of amide protons, acquiring MR image data of the region of interest, and assessing one of amide proton content, or pH in the region of interest using a ¹H saturation transfer technique. The method also includes adjusting contrast of the acquired MR image data based on said assessing of said one of amide proton content or pH so the adjusted acquired MR image data reflects relative differences of said one of amide proton content or pH within the region of interest. The imaging method can further comprises generating images based on the adjusted acquired MR image data. In particular embodiments, the exogenous contrast agent comprises one of one of a cationic polymer, a polymide (e.g., dendrimers, poly-lysines and polyglutamate), polyimino, poly-amino, or polyimine compounds.

According to yet another aspect of the present invention, there is featured a method of NMR including acquiring NMR image data that includes placing one of a sample or subject of interest in an NMR scanner, the sample or subject including an exogenous contrast agent there within, said contrast agent being characterized as being a source of amide protons, selectively exciting NMR signal in at least said contrast agent, and detecting signals from said contrast agent. Such a method also includes assessing one of amide proton content or pH based on the detected signals from said contrast agent using a ¹H saturation transfer technique and adjusting the generated NMR image data based on said assessing so the adjusted generated NMR image data reflects relative differences of said one of amide proton content or pH. In further embodiments, the contrasting agent comprises one of a cationic polymer, a polymide (e.g., dendrimers, poly-lysines and polyglutamate), polyimino, poly-amino, or polyimine compounds.

In further embodiments, said assessing includes irradiating a pool, an exogenous pool, of amide protons of said contrast agent that is in exchange with another pool of protons in said at least one region of said sample or subject with sufficient electromagnetic radiation to magnetically label the amide protons of said pool of amide protons and assessing said one of amide proton content, or pH based on transfer of saturation between said pool of amide protons and said another pool of protons.

According to another aspect of the present invention, there is featured a method for magnetic resonance imaging a molecular or cellular target within a body or sample. Such a method includes tagging the molecular or cellular target with a contrast agent, the contrast agent being characterized as being a source of amide protons and introducing the tagged molecular or cellular target into the body or sample (e.g., administering the tagged molecular or cellular target to the body of a patient by, for example by directing injection). Such a method also includes acquiring MR image data of the region of interest, assessing one of amide proton content, or pH in the region of interest using a ¹H saturation transfer technique; and determining the presence of the tagged molecular or cellular target within the region of interest based on said assessing.

The method further includes adjusting image data to localize the tagged molecular or cellular target so the target appears in the image generated from the image data. Also, the contrasting agent comprises one of a cationic polymer, a polymide (e.g., dendrimers, poly-lysines and polyglutamate), polyimino, poly-amino, or polyimine compounds.

According to another aspect of the present invention, there is featured a method for MR! NMR imaging delivery of a molecular or cellular target to a specified organ or tissue within a body. Such a method includes tagging the molecular or cellular target with a contrast agent, the contrast agent being characterized as being a source of amide protons and introducing the tagged molecular or cellular target into the body or sample. The method also inlcudes acquiring an MR image data set of the region of interest, assessing one of amide proton content, or pH in the region of interest using a ¹H saturation transfer technique, and determining the presence of the tagged molecular or cellular target within the region of interest based on said assessing. Also, said acquiring, said assessing and said determining are repeated so as to acquire a plurality of MR image data sets that are in a time sequence and so as to provide successive determinations of the presence of the tagged molecular or cellular target for each of the plurality of MR image data sets.

In further embodiments, the method further includes adjusting the image data of each of the plurality of MR image data sets so as to reflect a location of the tagged molecular or cellular target in each of the data sets and comparing each of the plurality of image MR data sets so as to establish a travel path of the tagged molecular or cellar target within the body. The molecular or cellular target is one of a gene, gene expressions, stem cell, antibody or therapeutic. Also, the contrast agent is further configured and arranged so as to be a carrier for said one of a gene, gene expressions, stem cell, antibody or therapeutic.

According to yet further aspects of the present invention there are featured a method for relating an amide proton exchange properties to cellular pH, and a method for imaging amide proton content and properties via exchange relationship of amide protons with the water signal.

The above-described methodology of the present invention can be adapted so the exogenous contrast agent used therewith can be used as cellular labels, MR signal enhancement agent, or as a carrier for one or more substances selected from receptor-binding of ligands, oligonucleotides, RNA, DNA, plasmids, or small molecule drugs.

The methods of the present invention advantageously increase the sensitivity of several protons of the cationic polymer in the gene delivery system to Magnetic Resonance Spectroscopic techniques, e.g., NMR, MRS, and MRI, by using the inherent properties of acidic protons present in the cationic polymer to enhance the signal sensitivity by factors of up to about 500,000 or more. The methods of the invention allow for the detection of micromolar concentrations of macromolecules having acidic protons with the molar sensitivity of water.

In summary, the methods of the present invention advantageously allow micromolcular concentrations of polymers, such as those described herein, to be detected by exploiting the molar sensitivity of water. It also is within the scope of the present invention for the foregoing methods to be adapted so as to be used with tailored design of a family of polyamide-based contrast agents that are optimized with respect to the number of selectively saturable exchange protons per molecular weight unit. It is further contemplated that the methods of the present invention be adapted such that the contrats agents include a maximum number of exchangeable protons in the correct pKa range so as to further provide an additional order of magnitude of enhancement.

Other aspects and embodiments of the invention are discussed below.

### BRIEF DESCRIPTION OF THE DRAWINGS:

For a fuller understanding of the nature and desired objects of the present invention, reference is made to the following detailed description taken in conjunction with the accompanying drawing figures wherein like reference character denote corresponding parts throughout the several views and wherein:

FIG. 1 illustrates the absorption line shapes for the protons in the macromolecular pool and the liquid pool;

FIG. 2 is a two-pool model of the magnetization transfer process; and

FIG. 3 is a series of NMR plots showing water attenuation due to selective radio frequency saturation as a function of chemical shift with respect to water, which is set at 0 ppm (z-spectrum). The curves for PAA and PEI are coincident and only one curve for PEI is displayed. The pulse sequence consisted of a continuous low-power rf saturation (500 MHz VARIAN spectrometer; tₛₐₜ=10 sec; power 100 Hz; interscan delay of 17 sec).

### DETAILED DESCRIPTION OF THE INVENTION

The present invention features an MRI/NMR methodology or process for detecting exogenous amide protons in a region of interest of a body or sample via the water signal. Such methods and processes can be used for any of a number of purposes including determining and assessing the delivery and/or content of a molecular or cellular target(s), such as ligands, oglionucleotides, and RNA/DNA (including plasmids) tagged or labeled by an exogenous contrast agent sourcing such amide protons; detecting and assessing pH effects, more particularly the pH of the liquid pool (e.g., blood); and as a mechanism for MR/NMR signal enhancement (e.g., providing another mechanism for developing contrast between tissues, etc. of the region of interest. According to various aspects of the present invention, also featured are methods whereby assessment of the delivery or the efficacy of delivery, pH effects or the signal enhancement can be used in connection with diagnosis and treatment of any of a number of diseases or disorders of the body, including but not limited to, brain related disorders and diseases, cardiac diseases and disorders, cancer, ischemia, Alziheimers, Parkinsons, and auto-immune diseases.

Before describing the present invention, the following briefly and generally describes the magnetization transfer process, where reference also should be made to U.S. Pat. No. 5,050,609 and to Magnetization Transfer in MRI: A Review infra, for further details or description of the magnetization transfer process. As indicated herein, coupling between the immobile, solid-like macromolecular protons and the mobile or “liquid” protons allows the spin state of the immobile macromolecular protons to influence the spins state of the liquid protons (e.g., water) through exchange processes. As is known in the art, it is possible to saturate the immobile macromolecular spins preferentially using an off-resonance radio frequency (RF) pulse. Such saturation also is referred to as magnetically labeling of the macromolecular protons. The immobile macromolecular spins have a much broader absorption lineshape than the liquid spins, making them as much as 10⁶ times more sensitive to an appropriately placed off-resonance RF irradiation. This saturation of the macromolecular spins is transferred to the liquid spins, depending upon the rate of exchange between the two spin populations, and hence is detectable with MRI.

There is shown in FIG. 2, a two-pool model that provides a quantitative interpretation of such magnetization or saturation transfer. Pool A represents the liquid spins, where the number of spins in this compartment is by convention normalized to unity (M_{OA}=1), and Pool B represents the macromolecular spins. In tissues, the number of immobile macromolecular spins is much less than the liquid spins and the relative fraction is given by M_{OB}. In each pool, and at any instant in time, some of the spins are in the longitudinal orientation represented by the upper unshaded portion of the compartment and some spins are saturated, represented by the lower shaded portion. The partition into longitudinal spins and saturated spins depends upon the irradiation history. When the irradiation is turned off, the time-dependent changes in the model are represented by rate constants, the longitudinal relaxation rates of pools A and B (R_{A} and R_{B}, respectively), the exchange rate from Pool A to Pool B (RM_{OB}) and the exchange rate from Pool B to Pool A (R).

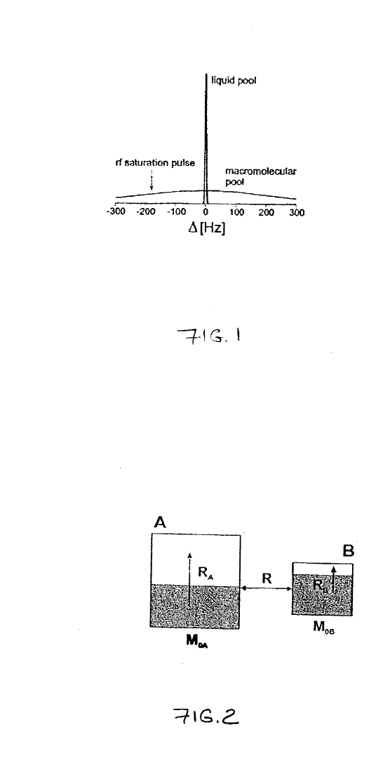

In Pool B, the protons in the macromolecules are strongly coupled to each other resulting in a homogenously broadened absorption lineshape as is shown in FIG. 1. Thus, the off-resonance irradiation results in progressive saturation of the spins that make-up Pool B. In contrast, the spins making up Pool A are weakly coupled due to motional narrowing. Although the intent with magnetization transfer is to manipulate the spins of the liquid pool indirectly by means of the saturating the macromolecular pool, some direct saturation of the liquid pool in Pool A is inevitable, which is generally described by the Bloch equations.

As indicated herein, the most important process in magnetization transfer is the exchange between the immobile macromolecular pool, Pool B, and the liquid pool, Pool A. It is this exchange that transfers the saturation or magnetization of the macromolecular protons to the protons comprising the liquid pool, which results in decreased longitudinal magnetization being available for imaging.

According to one aspect of the present invention, there is featured a method or process using MR or NMR techniques for imaging the delivery of a molecular or cellular target(s), such target(s) including but not limited to genes, gene expressions, antibodies or therapeutic agents, to a specific organ(s) or tissue(s). Such a method includes providing a delivery system, more particularly a non-viral delivery system, for the molecular or cellular target that includes an MRI/NMR contrast agent, the contrast agent being a compound or other formulation that provides a source of amide protons. In further embodiments, the contrast agent also comprises the carrier for the molecular or cellular target(s) or is bound to the molecular or cellular target(s) using any of a number of techniques known to those skilled in the art.

In particular embodiments, the contrast agent includes one of a cationic polymer, a polymide (e.g., dendrimers, poly-lysines and polyglutamate), polyimino, poly-amino, or polyimine compounds. In more particular embodiments, the contrast agent further comprises the carrier for receptor binding of ligands, oglionucleotides, and RNA/DNA (including plasmids).

In further particular embodiments, the contrast agent comprises a polymer having a plurality of functional groups capable of exchanging at least one amide proton with water and the plurality of functional groups have a resonance frequency different from the resonance frequency of water and which can be saturated by proton exchange between the functional group and water. In other embodiments, the functional group has one of a pKₐ in the range of between about 3 and about 5, a pKₐ in the range of between about 3.5 and about 4.5 or a pKₐ of about 4. Also, the functional group is selected from primary amides, primary amines, secondary amines, imines, imides, mono functional ureas, 1,3-difunctional ureas and combinations thereof. In yet further embodiments, there is one of at least one exchangeable protons per monomer repeat unit of the cationic polymer, at least two exchangeable protons per monomer repeat unit of the cationic polymer, at least two (2) exchangeable protons per kDalton in the cationic polymer, at least four (4) exchangeable protons per kDalton in the cationic polymer or at least ten (10) exchangeable protons per kDalton in the cationic polymer.

In further embodiments; prior to administration of the combined molecular/cellular target (s) and delivery system (hereinafter molecular/cellular complex), the MR/NMR imaging system applies a series of magnetic resonance pulses (radio frequency pulses) to a region of interest in the body or a sample. The detection system thereof measures or determines a baseline or pre-contrast response of the region of interest (e.g., artery and/or tissues in the region of interest) to that series of pulses. The series of magnetic resonance pulses are applied to the patient to tip the longitudinal magnetization of protons in the region of interest and to measure the response of the region of interest before administration of the contrast agent to the body or sample. The response signal from the region of interest is monitored using a variety of coils of an imaging coil apparatus and is measured by the detection system.

After such a baseline or pre-contrast response is measured, the combined molecular/cellular complex including the contrast agent is administered to the body or sample. Such administration is accomplished using any of a number of techniques known to those skilled in the art (e.g., direct injection into the body or via an IV). Thereafter, the detection system measures (continuously, periodically or intermittently) the response from the region of interest to detect the “arrival” of the contrast agent in the region of interest and thus the arrival also of the molecular/cellular constituent. The magnetic MRI system applies a series of magnetic resonance pulses and the detection system evaluates the response from the region of interest. When the contrast agent “arrives” in the region of interest (e.g., such as a specific organ or tissues of the of the body, an artery or arteries of interest), the detection system detects a characteristic change in the response from the region of interest to the water signal from the region of interest. This characteristic change in radio frequency signal from the region of interest indicates that the contrast agent has “arrived” in target region. The detector relays signal to the processor which initiates the process of data collection until an image is generated. However, in other embodiments, the processor collects data at predetermined intervals.

As to the detection of the “arrival” of the contrast agent in the region of interest and according to further embodiments, the methodology of the present invention detects the effects of amide proton properties, pH or pH effects on the intensity of the water signal in MRI. More particularly, according to the methodology and process of the present invention, the narrow amide proton resonance range of the material (e.g., compounds) comprising the exogenous contrast agent are selectively irradiated and saturated. The saturation is subsequently transferred to the water (¹H) protons as with the ¹H magnetization transfer process.

In more particular embodiments, the imaging apparatus is configured so as to be capable of selectively irradiating and saturating the amide proton resonance range of the exogenous amide protons (e.g., amide protons of the contrast agent) in the region of interest being imaged. The saturation is subsequently transferred to the water (¹H) protons in the region of interest as with the ¹H magnetization transfer process.

More specifically, the main amide proton resonance of the exogenous mobile protons (i.e., exogenous amide protons) centered around 8.3 ppm in the proton NMR spectrum for amide protons is selectively irradiated and saturated. Thereafter, using known MR imaging spectroscopy techniques (e.g., applying magnetic field gradients to spatially resolve the NMR signal intensity of the saturation transferred to the water protons) NMR data is obtained from such a signal(s) and such data is recorded for evaluation and assessment. In more particular embodiments, in accordance with the methodology of the present invention, the limited frequency range for mobile spectral macromolecular components (e.g., range of about 5-6 ppm wide, corresponding to 300-360 Hz wide at 1.5 Telsa, 600-720 Hz wide at 3 Telsa, etc.) is evaluated and assessed. This is different from the methodology of conventional MT that looks at a wide frequency range (e.g., several tens—hundreds of kHz) for the immobile, solid like components. In the procedure outlined, to determine the amide-proton transfer effect, the effect of conventional MT is removed and/or assessed so as to not be included or not to dominate.

Thereafter, an assessment is made from the recorded data as to the effect of the saturated amide protons of the exogenous contrast agent on the water signal. From this assessment a determination also is made as to the “arrival” or not of the contrast agent in the region of interest. In more particular embodiments, the method or process includes making a determination from the recorded data as to the amide proton transfer effect being exhibited and, based on the determined amide proton transfer effect, making a determination as to arrival or not of the contrast agent. In more particular embodiments, the amide proton transfer effect manifests itself as an amide proton transfer ratio and/or signal intensity of the amide protons. The amide proton transfer ratio as herein described depends upon amide content (intensity) and on the amide proton exchange rate. In addition, in the methodology of the present invention the effect of the conventional MT is eliminated or removed by assessing asymmetry and signal changes on top of this asymmetry.

Such a method further includes, comparing the acquired image data for each acquisition and assessing the movement within the region of interest of the body, of the contrast agent between successively acquired image data sets. In this way, the delivery of the molecular/cellular target(s) as a function of time and the efficacy of such delivery can be determined and assessed. The use of polyamides and other polymers with exchangeable protons, e.g., polyimines, polyimides, polyamines and the like, as herein described provides a mechanism for visualization of cellular or molecular targets using low concentrations of the polymer with exchangeable protons. These polymers allow for the use biological and biocompatible polymers as contrast agents during MRI and MRS visualization during delivery of a gene or other therapeutic agent to a target organ or tissue.

According to another aspect of the present invention there is featured a method or process for MR imaging that detects the effects of amide proton properties of the exogenous contrast agent, pH and/or the content (i.e., concentration) of the molecular cellular targert(s) on the intensity of the water signal in MRI. More particularly, according to the methodology and process of the present invention, the narrow amide proton resonance range of the exogenous contrast agent that sources such amide protons is selectively irradiated and saturated. The saturation is subsequently transferred to the water (¹H) protons as with the ¹H magnetization transfer process.

More specifically, the main amide proton resonance of the exogenous mobile protons centered around 8.3 ppm in the proton NMR spectrum for amide protons is selectively irradiated and saturated. Thereafter, using known MR imaging/spectroscopy techniques (e.g., applying magnetic field gradients to spatially resolve the NMR signal intensity of the saturation transferred to the water protons) NMR data is obtained from such a signal(s) and such data is recorded for evaluation and assessment. It more particular embodiments, in accordance with the methodology of the present invention, the limited frequency range for mobile spectral macromolecular components (e.g., range of about 5-6 ppm wide, corresponding to 300-360 Hz wide at 1.5 Telsa, 600-720 Hz wide at 3 Telsa, etc.) is evaluated and assessed. This is different from the methodology of conventional MT that looks at a wide frequency range (e.g., several tens—hundreds of kHz) for the immobile, solid like components. In the procedure outlined, to determine the amide-proton transfer effect, the effect of conventional MT is removed and/or assessed so as to not be included or not to dominate.

Thereafter, an assessment is made from the recorded data as to the effect of the saturated amide protons on the water signal. From this assessment a determination also is made as to the amide proton content, content/concentration of the exogenous contrast agent and/or the content/concentration of the molecular/cellular target(s) associated therewith, and/or pH. In more particular embodiments, the method or process includes making a determination from the recorded data as to content/concentration of the exogenous contrast agent and/or the content/concentration of the molecular/cellular target(s) associated therewith, and/or pH.

In more specific embodiments, the method or process of the present invention further includes establishing a relationship between amide proton transfer effect and the characteristic, for example pH, to be determined and using the relationship in combination with the determined amide proton transfer effect, making a determination as to the amide proton content, the content or concentration of the exogenous material sourcing the amide protons and/or pH. In more particular embodiments, the amide proton transfer effect manifests itself in the form of one or an amide proton transfer ratio and/or a signal intensity of the amide protons. In addition, in the methodology of the present invention, the effect of conventional MT is eliminated or removed by assessing MT asymmetry and signal changes on top of this asymmetry.

According to yet another aspect of the present invention there is featured a method or process for magnetic resonance imaging where the spatial information comprising the image data is obtained by combining the methodology or process for MR imaging that detects the effects, more particularly the relative effects, of amide proton content and/or pH on the intensity of the water signal in MRI along with any water imaging (MRI) approach and any spectroscopic imaging methodology (e.g., one-dimensional and/or multi-directional phase encoding with pulsed field gradients). In this way, the image data is adjusted so as to further reflect at least the relative effects or differences of amide proton content or pH of the tissues and/or bodily fluids being imaged. Stated another way, the contrast of the image data is adjusted or modified so as to further reflect at least the relative effects or differences of amide proton content/properties or pH of the tissues and/or bodily fluid being imaged. Thus, the diagnostic images being generated from the so-adjusted or modified image data provide further contrast between tissues and/or bodily fluids having different amide proton content/properties and/or pH.

As is known in the art, body tissue that has experienced trauma or infarct, cancerous tissues, whether benign or malignant, or other insult typically has different physiological and chemical characteristics than that for normal tissue that surround the insulted body tissue. Thus, adjusting the contrast for MR images to reflect the relative amide proton content and properties or relative pH of the various tissues or bodily fluids of the region of interest being imaged advantageously enhances the MR imaged being generated so as to provide further contrast between normal tissue and the tissue experiencing the insult.

In more particular embodiments, before or after acquiring the NMR/MR image data using known imaging techniques, the imaging apparatus is configured so as to be capable of selectively irradiating and saturating the amide proton resonance range of exogenous amide protons (e.g., amide protons of the exogenous contrast agent) in the region of interest being imaged. The saturation is subsequently transferred to the water (¹H) protons in the region of interest as with the ¹H magnetization transfer process. More specifically, the amide proton resonance(s) of the amide protons of the exogenous contrast agent centered around 8.3 ppm in the proton NMR spectrum for amide protons are selectively irradiated and saturated. Thereafter, using known MR imaging spectroscopy techniques (e.g., applying magnetic field gradients to spatially resolve the NMR signal intensity of the saturation transferred to the water protons) NMR data is obtained from such a signal(s) and such data is recorded for evaluation and assessment.

Thereafter, an assessment is made from the recorded data as to the effect of the saturated amide protons on the water signal. From this assessment a determination also is made as to amide proton content and properties, and/or the pH and/or pH changes. In a further embodiment, an assessment is made to determine or establish a relative difference between the amide proton content and properties, and/or the pH of the cells of the tissues in the region of interest. For example, the in-process values that are representative of the characteristic being determined (e.g., pH) can be normalized and the normalized values used to adjust the image data or the contrast of the image data.

In another more particular embodiment, the method or process includes making a determination from the recorded data as to the amide proton transfer effect being exhibited by the various tissues of the region of interest and, based on the determined amide proton transfer effect, determining or establishing the relative difference between the exogenous amide proton content and properties, and/or the pH. As indicated above, these in process values of amide proton transfer effects can be normalized and the normalized values used to adjust the image data or the contrast of the image data.

In still another further particular embodiment, the method or process includes making a determination from the recorded data as to the amide proton transfer effects being exhibited and, based on the determined amide proton transfer effect, making a determination as to the exogenous amide proton content and properties and/or the pH. In more specific embodiments, the method or process of the present invention further includes establishing a relationship between amide proton intensity and/or transfer rates and the sought characteristic, for example, amide proton content and/or pH. After making such determination as to the exogenous amide proton content and properties, and/or pH, the image data is adjusted, more specifically the contrast of the tissue and/or bodily fluids within the region of interest is adjusted based on the determined exogenous amide proton content and properties, and/or the pH of the cells.

According to yet another further particular embodiment, the method or process of the present invention further includes establishing a relationship, more specifically an empirical relationship, between an amide proton transfer effect, more specifically between amide proton intensity and/or amide proton transfer ratios, and the sought characteristic or property, for example, amide proton content and/pH. In more specific embodiments, such establishing of a relationship is accomplished in vivio, using tissues extracted from the area of interest or using a sample having pre-determined characteristics.

In an exemplary illustrative embodiment, the sought characteristic is tissue/cellular and/or bodily fluid pH and said establishing a relationship includes establishing an empirical relationship between the amide proton transfer effect of the amide protons and such pH. Such a method is accomplished by irradiating a first pool including the amide protons, that is in exchange with a second pool of protons, with sufficient electromagnetic radiation to label the amide protons of said first pool and determining a given amide proton transfer effect corresponding to the transfer of saturation between said first pool of amide protons and said second pool of protons. In the present invention, the first pool of protons comprises amide protons of the contrast agent.

A phosphorus spectroscopy also is performed to determine a cellular pH value corresponding to the determined amide proton transfer ratio. These steps of irradiating, determining and performing the phosphorous spectroscopy are repeated for several physiological conditions (e.g., several different pH conditions) so as to generate a pH values corresponding to respective determined amide proton transfer ratio ; and the empirical relationship is created using the generated plurality of pH values corresponding to respective determined amide proton transfer effects. In more specific embodiments, the amide proton transfer effect comprises an amide proton transfer ratio and the pool of amide protons is from the exogenous contrast agent.

According to further embodiments of the present invention, the MR/NMR imaging is imaging an intravascular feature of a body and such a MRI technique includes inserting a novel loopless antenna into vessels (Ocali and Atalar, 1997, MRM 37:112-118). Using this particular technique, high-resolution MR images of arterial walls and atherosclerotic plaques can be obtained. The acquisition of real-time MR fluoroscopic images can be used to guide intravascular interventions (see, e.g., Correia, et al., 1997, Arterioscler. Thromb. Vasc. Biol. 17: 3626-2632; Yang and Atalar, 1999, Circulation 100: 1-799; Yang and Atalar, 2000, Radiology 217: 501-506; Yang, et al., 2001, Circulation 104: 1588-1590.

The following example(s), further illustrate the various methodologies and processes of the present invention. As this example is illustrative, the method and process of the present invention shall not be particularly limited to the following examples.

### EXAMPLE

Cationic polymers (CPs) have become increasingly important as nonviral DNA delivery systems for potential use in gene therapy. As such it would be useful if low concentrations of these compounds could be detected with sufficient sensitivity to allow non-invasive visualization of gene delivery or antibody targeting in vivo. Using current MRI techniques, it has been necessary to label these compounds, e.g., the cationic polymer or DNA for delivery, with at least one (super)paramagnetic tag.

The MR signal enhancement resulting from the methodology of the present invention, provides greater increases in signal enhancement using cationic polymers which contain a plurality of protons having a similar resonance frequency, i.e., chemical shift (δ). Because such protons can be simultaneously saturated, their total effective molarity is much higher than that of the molecule itself, allowing for the polymer to act as a saturation amplifier. For fast exchange relative to the longitudinal relaxation e.g., exchange between an amide and water is faster than longitudinal relaxation of the amide proton (k>>1/T_{1NH}), it can be derived that the proton-transfer enhancement is represented by the equation of Formula I:

$$PTE = {\sum }_{i} \frac{{α}_{i} {k}_{i} {N}_{i} {M}_{W}}{( 1 - {x}_{CP} ) {r}_{1 wat} + {x}_{CP} {k}_{i}} ( 1 - {ⅇ}^{- [ ( 1 - {x}_{CP} ) {R}_{1 wat} + {x}_{CP} {k}_{i} ] {t}_{sat}} )$$

where

α is the saturation efficiency (0<α<1);

k is the pseudo-first-order forward rate contstant;

N is the number of exchangeable protons of a particular type per molecular weight unit;

M_{W} is the molecular weight of the cationic polymer;

x_{CP} is the fractional concentration of exchangeable protons for the CP;

the exponential term descries the influence of back-exchange and the longitudinal relaxation rate (R_{1wat}=1/T_{wat}) of water protons on the buildup of this effect during the length of the saturation period (tₛₐₜ); and

i is the summation index over the different types of macromolecular NH protons having substantially similar chemical shifts (δ), e.g., amide protons, primary amine protons, and secondary amine protons having similar chemical shifts, but may have differing rates of exchange with water (kᵢ).

As is known in the polymer sciences the number of exchangeable protons in a polycation polymer or polyanion polymer or dendrimer is dependent on the monomer repeat units from which the polymer or dendrimer are composed as well as the architecture of the polymer or dendrimer. In a non-limiting illustrative example, different generations of a starburst polyamide dendrimer, PAMAM, (SPD-g; polymer XXX) has different numbers of exchangeable protons including one NH₂ group per surface group and 2s-4 extra amide protons in the individual branches of the dendrimer (where the number of surface groups, s, is 2^{g+2} and g is the generation number). Thus the total number of exchangeable protons is the sum of the number of surface protons and the number of internal amide protons. Thus, for a fifth generation PAMAM dendrimer of polymer X (SPD-5), which has 256 surface primary amine protons (s=2⁵⁺²; two protons per surface NH₂ group) and 252 internal amide groups (252=2s-4) for 508 total exchangeable protons in the fifth generation starburst dendrimer.

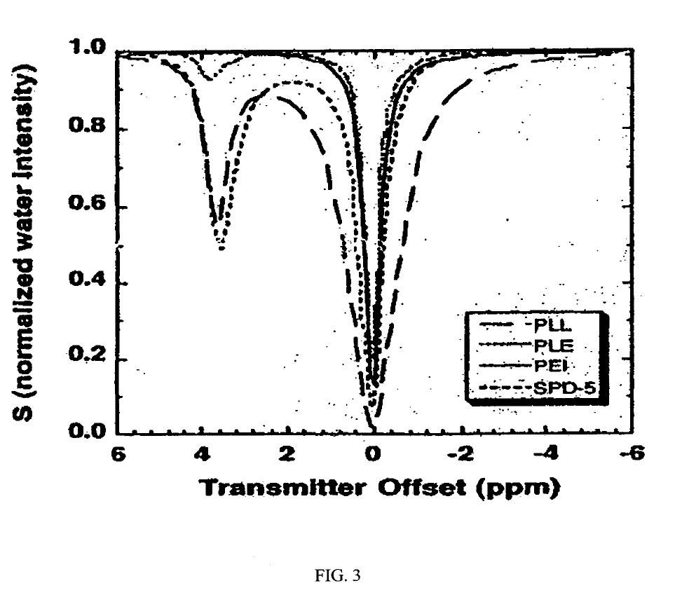

The proton transfer enhancement (PTE) of signal saturation transfer from the cationic polymer to water was determined for several cationic polymers (See Table 1). Samples were prepared in aqueous solution (95% 0.01 M phosphate buffered saline (PBS), 5% deuterium oxide by volume) at concentrations set to keep x_{CP} of detectable exchangeable protons similar between samples. To visualize the saturation transfer effect for the exchangeable protons, z-spectra (Annu. Rev. Biophys. Biomol. Struct. (1996) 25:29-53) or CEST-spectra (J. Magn. Reson. (2000) 143:79-87) was acquired, in which the reduction in the water signal due to saturation transfer is measured as a function of NMR frequency offset. In z-spectra, the reference frequency for water is set at 0 ppm, which corresponds to direct saturation of water. If at any frequency there are exchangeable protons at appropriate concentration and exchange rate, the effect becomes visible through attenuation of the water line. The resulting z-spectra in FIG. 3 show no noticeable saturation transfer effect for PPA or PEI while effects for different magnitude are measured for PLL, PLE and SPD-5.

The data presented in Table 1 indicates that only the amide protons are in the appropriate pKₐ range to be visible in the NMR spectrum as a separate resonance. Preferred protons for exchange have a pKₐ of between about 3 and about 5, more preferably between about 3.5 and about 4.5. Particularly preferred functional groups having exchangeable protons have a pKₐ of about 4. This feature of exchanging sufficiently slowly on the NMR timescale is a principal requirement for the methods of detecting macromolecules provided by the present invention. When proton exchange is too fast, a single resonance that is fractionally weighted between the chemical shifts of the exchange sites will be found, coinciding with water, and not targeted detection is possible. Also, exchange should be slow enough to allow sufficient saturation of NH protons before exchange. NMR visibility for the CP protons was checked using a flip-back approach to acquire spectra in which exchangeable protons are not suppressed. See, Magn. Reson. Med. (1998) 40:36-42 and J. Magn. Reson. (1996) 110:96-101, for the flip-back procedure. Measurable exchangeable protons were only observed for PLL, PLE, and SPD-5 using the flip-back approach. When integrating the peak areas and using the aliphatic protons as intensity reference, the intensity of the exchangeable protons agrees with that expected for the amide groups. This pKₐ limitation needs to be taken into account when designing proton-exchange-based contrast agents.

Saturation effects were measured independently of the shape of the water line by taking the ratio of the water signal intensity with (Sₛₐₜ) and without (S₀) saturation of the exchangeable groups, using the opposite side of the water line as reference for intensity. The resulting ratio should be related to the PTE via the following equation:

$$( 1 - \frac{{S}_{sat}}{{S}_{0}} ) = \frac{[ contrastagent ] \cdot PTE}{2 \cdot [ {H}_{2} O ]}$$

The data in Table 1 shows good agreement between calculate and observed effects. The reason that the water intensity reductions for SPD and PLL are comparable, despite the fact that the exchange rate for PLL (140 sec⁻¹) is much larger, is that the back-exchange from saturated water to the PLL is significant. The fact that the signal reduction is still overestimated by about 20% may be due to exchange being too fast to allow full saturation before exchange, thereby reducing a (assumed to be 1). The underestimation of the SPD signal reduction is attributed to the fact that the actual exchange rate may be larger than the measured value. NMR spectra acquired at lower pH show that there are three different amide groups that partially overlap in chemical shift in the NMR spectrum, each of which has a different exchange rate that contributes to the PTE value of the dendrimer. However at physiological pH it is difficult to resolfve the broad signals and to determine the individual exchange rates.

For methods of the present invention of detecting or imaging of cationic polymers in vivo, the asymmetry of the z-spectrum for exchangeable protons is used to separate the CP effect from the magnetization transfer contrast (MTC) z-spectrum, which is approximately symmetric. MTC and direct water saturation are separate from but additional to the exchange effect, and saturation power should be optimized to minimize these effects with respect to exchange transfer. This is expected to be accomplished with saturation powers that are less than for MTC. High magnetic fields are beneficial for this new contrast mechanism, because the amide protons are better resolved and T_{1wat} is longer than at low field. For instance, T_{1wat} in vivo is about 1 s at 1.5 T, leading to effects that are about 30% to about 40% of the effects measured at 11.7 T.

Chart 1. Structural formula of various ionic polymers:

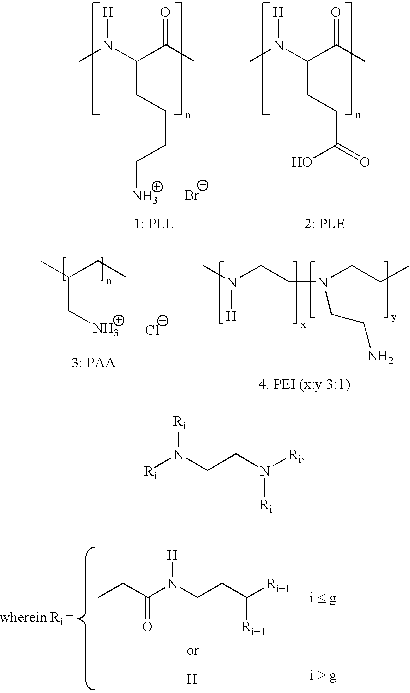

5: Starburst™ PAMAM dendrimer

PLL is intended to refer to poly-L-lysine

PLE is intended to refer to poly-L-glutamate

PAA is intended to refer to polyallylamine

PEI is intended to refer to polyetheylenimine

TABLE 1
Cationic Polymer Data and Results for Saturation Transfer and Exchange Properties
(pH 7.3-7.4, T = 37° C.)^{a}

| | | | | | | | | | (S_{o -} | |
|---|---|---|---|---|---|---|---|---|---|---|
| | | | N (amide) | N (NH)^{b} | N(NH₂) | | | | Sₛₐₜ)/S_{O} | |
| | | Conc. | protons/k | protons) | protons/k | k^{d} | | | obsd | |
| | M_{w} kD | (μM) | D | kD | D | (s⁻¹⁾ | Xcp × 10³ | PTE | calcd^{f} | |
| PLL | 488 | 100 | 4.78 | 0 | 9.57^{c} | 140 | 2.11 | 586,31 | 0.43 | 0.53 |
| PLE | 70 | 500 | 6.62 | 0 | 0 | 10 | 2.10 | 15,568 | 0.07 | 0.07 |
| PAA | 70 | 300 | 0 | 0 | 21.61^{c} | c | N/A | c | 0 | 0 |
| PEI. | 750 | 150 | 0 | 4.64^{c} | 9:29^{c} | c | N/A | c | 0 | 0 |
| SPD-5 | 28.825 | 1000 | 8.74 | 0 | 8.88^{c} | 77^{e} | 2.29 | 44,080 | 0.51 | 0.40 |

″Abbreviations: poly-L-lysine (PLL), poly-L-glutamate (PLE), polyallylamine (PAA), polyethylenimine (PEI), Starburst PAMAM dendrimers (SPD-5).
^{b}Nonamide-NH protons
^{c}Exchangeable protons not detectable in spectrum
^{d}Measured with the WEX-filter^{8b} approach.
^{e}Wide resonance containing multiple amide protons with different exchange rates.
^{f}Using eq 2, α = 1, and T_{1 wat} = 3.86 s (determined using an inversion recovery experiment).

Although a preferred embodiment of the invention has been described using specific terms, such description is for illustrative purposes only, and it is to be understood that changes and variations may be made without departing from the spirit or scope of the following claims. All references cited herein are incorporated by reference into the present application.

What is claimed is:

1. A method for determining an effect of amide proton content and properties of an exogenous contrast agent on a water signal as measured by one of MRI or NMR spectroscopy or spectroscopic imaging, said exogenous contrast agent being configured and arranged so as to provide a pool of amide protons that is in exchange with another pool of protons; said method comprising the steps of:
irradiating said pool of amide protons that is in exchange with said another pool of protons to label the amide protons of said pool of amide protons and measuring the effect on the protons the amide protons are in exchange with;
determining an amide proton transfer effect corresponding to the transfer of saturation between said pool of amide protons and said another pool of protons; and
determining one of amide proton content, pH or pH effects from the determined amide proton transfer effect.

2. The method of claim 1, wherein said exogenous contrast agent comprises one of one of a cationic polymer, a polymide including dendrimers, poly-lysines and polyglutamate, polyimino, poly-amino, or polyimine compounds.

3. The method of claim 2, wherein said another pool of protons comprises water.

4. The method of claim 1, wherein said determining an amide proton transfer effect includes determining one of an amide proton transfer ratio, an amide proton transfer rate or an amide proton signal intensity.

5. The method of claim 1, wherein said irradiating includes irradiating the amide protons at a resonance in a proton spectrum of the amide protons.

6. The method of claim 1, wherein said irradiating includes irradiating the amide protons with electromagnetic radiation at about a 8.3 ppm resonance in a proton spectrum of the amide protons.

7. The method of claim 1, wherein said irradiating includes irradiating the amide protons with electromagnetic radiation around a 8.3 ppm resonance in a proton spectrum of the amide protons.

8. The method of claim 1, wherein determining an amide proton transfer effect includes magnetic resonance imaging of the second pool of protons a predetermined period of time after transfer of saturation.

9. The method of claim 1, further comprising the step of establishing a relationship between proton transfer effect of amide protons and said one of amide proton content, pH or pH effects.

10. The method of claim 7, wherein said establishing a relationship includes establishing an empirical relationship between the proton transfer effect of amide protons and said one of amide proton content, cellular pH or pH effects.

11. The method of claim 10, wherein said establishing an empirical relationship includes establishing an empirical relationship between the proton transfer effect of amide protons and pH including:
irradiating a first pool including amide protons of the contrast agent, that is in exchange with a second pool of protons, with sufficient electromagnetic radiation to label the amide protons of said first pool;
determining a given amide proton transfer effect corresponding to the transfer of saturation between said first pool of amide protons and said second pool of protons;
performing a phosphorus spectroscopy to determine a cellular pH value corresponding to the determined amide proton transfer effect;
repeating said steps of irradiating, determining and performing so as to generate a plurality of tissue pH values corresponding to respective determined amide proton transfer effects; and
creating said empirical relationship using the generated plurality of tissue pH values corresponding to respective determined amide proton transfer rates.

12. A method for magnetic resonance imaging comprising the steps of:
locating a contrast agent within a region of interest for a body or sample, the contrast agent being characterized as being a source of amide protons;
acquiring MR image data of the region of interest;
assessing one of amide proton content, or pH in the region of interest using a ¹H saturation transfer technique; and
adjusting contrast of the acquired MR image data based on said assessing of said one of amide proton content or pH so the adjusted acquired MR image data reflects relative differences of said one of amide proton content or pH within the region of interest.

13. The imaging method of claim 12, wherein said contrasting agent comprises one of a cationic polymer, a polymide including dendrimers, poly-lysines and polyglutamate, polyimino, poly-amino, or polyimine compounds.

14. The imaging method of claim 12, further comprising the step of:
generating images based on the adjusted acquired MR image data.

15. The imaging method of claim 12, wherein said assessing includes:
irradiating a pool of amide protons of said contrast agent in the region of interest that is in exchange with another pool of protons in the region of interest with sufficient electromagnetic radiation to label the amide protons of said pool of amide protons; and
assessing said one of amide proton content, or pH based on transfer of saturation between said pool of amide protons and said another pool of protons.

16. The imaging method of claim 12, wherein said assessing further includes:
irradiating a pool of amide protons of said contrast agent in the region of interest that is in exchange with another pool of protons in the region of interest with sufficient electromagnetic radiation to magnetically label the amide protons of said pool of amide protons; and
determining a given amide proton transfer effect corresponding to the transfer of saturation between said pool of amide protons and said another pool of protons; and
assessing said one of amide proton content, or pH based on the determined given amide proton transfer effect.

17. The method of claim 16, wherein:
said assessing includes assessing amide proton content based on the determined given amide proton transfer effect; and
said adjusting includes adjusting the contrast of the acquired MR image data based on said assessing amide proton content so the adjusted acquired MR image data reflects the relative differences in amide proton content.

18. The method of claim 16, wherein:
said assessing includes assessing pH based on the determined given amide proton transfer effect; and
said adjusting includes adjusting the contrast of the acquired MR image data based on said assessing pH so the adjusted acquired MR image data reflects the relative differences in pH.

19. A method of NMR comprising the steps of:
acquiring NMR image data that includes:
placing one of a sample or subject of interest in an NMR scanner, the sample or subject including an exogenous contrast agent there within, said contrast agent being characterized as being a source of amide protons;
selectively exciting NMR signal in at least said contrast agent, and
detecting signals from said contrast agent;
assessing one of amide proton content or pH based on the detected signals from said contrast agent using a ¹H saturation transfer technique; and
adjusting the generated NMR image data based on said assessing so the adjusted generated NMR image data reflects relative differences of said one of amide proton content or pH.

20. The NMR method of claim 19, wherein said contrasting agent comprises one of a cationic polymer, a polymide including dendrimers, poly-lysines and polyglutamate, polyimino, poly-amino, or polyimine compounds.

21. The NMR method of claim 19, wherein said assessing includes:
irradiating a pool of amide protons of said contrast agent that is in exchange with another pool of protons in said at least one region of said sample or subject with sufficient electromagnetic radiation to magnetically label the amide protons of said pool of amide protons; and
assessing said one of amide proton content, or pH based on transfer of saturation between said pool of amide protons and said another pool of protons.

22. The NMR method of claim 19, wherein said assessing further includes:
irradiating an exogenous pool of amide protons in said at least one region of said sample or subject that is in exchange with another pool of protons in said at least one region of said sample or subject with sufficient electromagnetic radiation to magnetically label the amide protons of said pool of amide protons; and
determining a given amide proton transfer effect corresponding to the transfer of saturation between said exogenous pool of amide protons and said another pool of protons; and
assessing one of amide proton content or pH based on the determined given amide proton transfer effect.

23. The NMR method of claim 19, wherein said adjusting includes adjusting the contrast of the generated NMR image data based on said assessing of amide proton content so the adjusted NMR image data reflects the relative differences in amide proton content.

24. The NMR method of claim 19, wherein said adjusting includes adjusting the contrast of the generated NMR image data based on said assessing of pH so the adjusted NMR image data reflects the relative differences in pH.

25. A method for relating amide proton exchange properties to tissue pH, comprising the steps of:
providing an exogenous contrast agent, said exogenous contrast agent being configured and arranged so as to provide a pool of amide protons that is in exchange with another pool of protons;
irradiating said pool of amide protons that is in exchange with said another pool of protons to label the amide protons of said pool of amide protons and measuring the effect on the protons the amide protons are in exchange with;
determining an amide proton transfer effect corresponding to the transfer of saturation between said pool of amide protons and said another pool of protons; and
determining tissue pH from the determined amide proton transfer effect.

26. The NMR method of claim 28, wherein said contrasting agent comprises one of a cationic polymer, a polymide including dendrimers, poly-lysines and polyglutamate, polyimino, poly-amino, or polyimine compounds.

27. The method of claim 25, further comprising the step of establishing a relationship between proton transfer effect of the amide protons and tissue pH.

28. The method of claim 25, wherein said establishing a relationship includes establishing an empirical relationship between the proton transfer effect of amide protons and tissue pH.

29. The method of claim 28, wherein said establishing an empirical relationship includes establishing an empirical relationship between the proton transfer effect of amide protons and tissue pH including:
irradiating a first pool including amide protons of said contrast agent, that is in exchange with a second pool of protons, with sufficient electromagnetic radiation to label the amide protons of said first pool;
determining a given amide proton transfer effect corresponding to the transfer of saturation between said first pool of amide protons and said second pool of protons;
performing a phosphorus spectroscopy to determine a pH value corresponding to the determined amide proton transfer effect;
repeating said steps of irradiating, determining and performing so as to generate a plurality of tissue pH values corresponding to respective determined amide proton transfer effects; and
creating said empirical relationship using the generated plurality of tissue pH values corresponding to respective determined amide proton transfer effects.

30. A method for imaging amide proton content and properties via exchange relationship of amide protons of an exogenous contrast agent with the water signal, said exogenous contrast agent being configured and arranged so as to provide a pool of amide protons that is in exchange with another pool of protons; said method comprising the steps of:
irradiating the exogenous pool of amide protons that is in exchange with said another pool of protons to label the amide protons of said exogenous pool of amide protons and measuring the effect on the protons the amide protons are in exchange with;
determining an amide proton transfer effect corresponding to the transfer of saturation between said pool of amide protons and said another pool of protons; and
determining one of amide proton content, cellular pH or pH effects from the determined amide proton transfer effect.

31. The method of claim 30, wherein said contrasting agent comprises one of a cationic polymer, a polymide including dendrimers, poly-lysines and polyglutamate, polyimino, poly-amino, or polyimine compounds.

32. The method of claim 30, wherein said another pool of protons comprises water.

33. The method of claim 30, wherein said irradiating includes irradiating the amide protons at a resonance in a proton spectrum of the amide protons.

34. The method of claim 30, further comprising the step of establishing a relationship between proton transfer effect and said one of amide proton content, tissue pH or pH effects.

35. The method of claim 34, wherein said establishing a relationship includes establishing an empirical relationship between the proton transfer effect and said one of amide proton content, tissue pH or pH effects.

36. The method of claim 35, wherein said establishing an empirical relationship includes establishing an empirical relationship between the proton transfer effect of amide protons and pH including:
irradiating a first pool including amide protons of said exogenous contrast agent, that is in exchange with a second pool of protons, with sufficient electromagnetic radiation to label the amide protons of said first pool;
determining a given amide proton transfer effect corresponding to the transfer of saturation between said first pool of amide protons and said second pool of protons;
performing a phosphorus spectroscopy to determine a pH value corresponding to the determined amide proton transfer effect;
repeating said steps of irradiating, determining and performing so as to generate a plurality of tissue pH values corresponding to respective determined amide proton transfer effects; and
creating said empirical relationship using the generated plurality of pH values corresponding to respective determined amide proton transfer effects.

37. The method of claim 36, wherein said repeating includes repeating said steps of irradiating, determining and performing for different physiological conditions.

38. A method for magnetic resonance imaging a molecular or cellular target within a body or sample, comprising the steps of:
tagging the molecular or cellular target with a contrast agent, the contrast agent being characterized as being a source of amide protons,
introducing the tagged molecular or cellular target into the body or sample;
acquiring MR image data of the region of interest;
assessing one of amide proton content, or pH in the region of interest using a ¹H saturation transfer technique; and
determining the presence of the tagged molecular or cellular target within the region of interest based on said assessing.

39. The method of claim 38, further comprising the step of adjusting image data to localize the tagged molecular or cellular target.

40. The method of claim 39, further comprising the step of adjusting contrast of the acquired MR image data based on said assessing of said one of amide proton content or pH so the adjusted acquired MR image data reflects relative differences of said one of amide proton content or pH for the tagged molecular or cellular target.

41. The method of claim 39, wherein said contrasting agent comprises one of a cationic polymer, a polymide including dendrimers, poly-lysines and polyglutamate, polyimino, poly-amino, or polyimine compounds.

42. A method for MR/NMR imaging delivery of a molecular or cellular target to a specified organ or tissue within a body, said method comprising the steps of:
tagging the molecular or cellular target with a contrast agent, the contrast agent being characterized as being a source of amide protons;
introducing the tagged molecular or cellular target into the body or sample;
acquiring an MR image data set of the region of interest;
assessing one of amide proton content, or pH in the region of interest using a ¹H saturation transfer technique;
determining the presence of the tagged molecular or cellular target within the region of interest based on said assessing; and
repeating said acquiring, said assessing and said determining so as to acquire a plurality of MR image data sets that are in a time sequence and so as to provide successive determinations of the presence of the tagged molecular or cellular target for each of the plurality of MR image data sets.

43. The method of claim 42, further comprising the step of adjusting the image data of each of the plurality of MR image data sets so as to reflect a location of the tagged molecular or cellular target in each of the data sets.

44. The method of claims 43, further comprising the steps of comparing each of the plurality of image MR data sets so as to establish a travel path of the tagged molecular or cellar target within the body.

45. The method of claim 42, wherein said contrasting agent comprises one of a cationic polymer, a polymide including dendrimers, poly-lysines and polyglutamate, polyimino, poly-amino, or polyimine compounds.

46. The method of claim 42, wherein the molecular or cellular target is one of a gene, gene expressions, stem cell, antibody or therapeutic.

47. The method of claim 46, wherein said contrast agent is further configured and arranged so as to be a carrier for said one of a gene, gene expressions, stem cell, antibody or therapeutic.

48. The method of claim 42, wherein said contrast agent comprises a polymer having a plurality of functional groups capable of exchanging at least one amide proton with water.

49. The method of claims 48, wherein the polymer comprises a plurality of functional groups having a resonance frequency different from the resonance frequency of water and which can be saturated by proton exchange between the functional group and water.

50. The method of claim 48, wherein the functional group has one of a pKₐ in the range of between about 3 and about 5, a pKₐ in the range of between about 3.5 and about 4.5 or a pKₐ of about 4.

51. The method of claim 48, wherein the functional group is selected from primary amides, primary amines, secondary amines, imines, imides, mono functional ureas, 1,3-difunctional ureas and combinations thereof.

52. The method of claim 45 wherein there is one of at least one exchangeable protons per monomer repeat unit of the cationic polymer, at least two exchangeable protons per monomer repeat unit of the cationic polymer, at least two (2) exchangeable protons per kDalton in the cationic polymer, at least four (4) exchangeable protons per kDalton in the cationic polymer or at least ten (10) exchangeable protons per kDalton in the cationic polymer.
